(19) United States
(12) Patent Application Publication
(10) Pub. No.: US 2009/0111069 A1
(43) Pub. Date: Apr. 30, 2009

(54) Tongue scraper and suction device

(76) Inventor: Anneke Wagner, Racine, WI (US)

Correspondence Address: RYAN KROMHOLZ & MANION, S.C., POST OFFICE BOX 26618, MILWAUKEE, WI, 53226, US

(21) Appl. No.: 12/231,808

(22) Filed: Sep. 5, 2008

Related U.S. Application Data
(60) Provisional application No. 60/995,663, filed on Sep. 27, 2007.

(51) Int. Cl. A61C 17/00; A61B 17/24

(52) U.S. Cl. 433/95; 433/91; 606/161

(57) ABSTRACT

A tongue scraper comprising a tongue scraping portion and a hand piece connected to the tongue scraping portion. A fluid passageway is located in the hand piece, which is connected to an external vacuum device.

### RELATED APPLICATION

The present application claims priority to provisional patent application, U.S. Ser. No. 60/995,663, filed on 27 Sep. 2007.

### BACKGROUND OF THE INVENTION

The present invention relates generally to devices for promoting dental hygiene, and relates more specifically to a tongue scraper used in combination with a suction device.

Tongue scrapers are used for cleansing food debris, volatile sulfur compounds, dead cells, microflora and other microorganisms from the tongue. There are a wide variety of tongue scrapers. Many tongue scrapers are designed as dual purpose devices, generally designed as a combination with a toothbrush or similar dental device. Such devices are sold to individuals and are generally used for personal hygiene.

However, it is also contemplated that dentists and hygienists may desire to use a tongue scraper when performing dental procedures, such as cleaning procedures and the like. When such procedures are performed, the dentist will normally use water and/or other fluids to irrigate the mouth when performing a procedure. When fluid is used, a suction device is also used to remove the fluid. However, space in the mouth is limited and using all of these instruments simultaneously can be cumbersome. It would be advantageous to have a device that could simplify such a tongue cleaning procedure.

### SUMMARY OF THE INVENTION

The present invention comprises a combination tongue scraper and suction device. The device generally consists of hand piece connected to a tongue scraping device. The hand piece has a fluid passageway that is connected to a vacuum device.

### BRIEF DESCRIPTION OF THE DRAWINGS

FIG. 1 is a perspective view of a device according to the present invention.

FIG. 2 is a cross-sectional view of the device of FIG. 1.

FIG. 3 depicts the device of the present invention being used within the mouth of a person.

FIG. 4 is a perspective view of an alternate embodiment of a device according to the present invention.

FIG. 5 is a cross-sectional view of the embodiment shown in FIG. 4.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

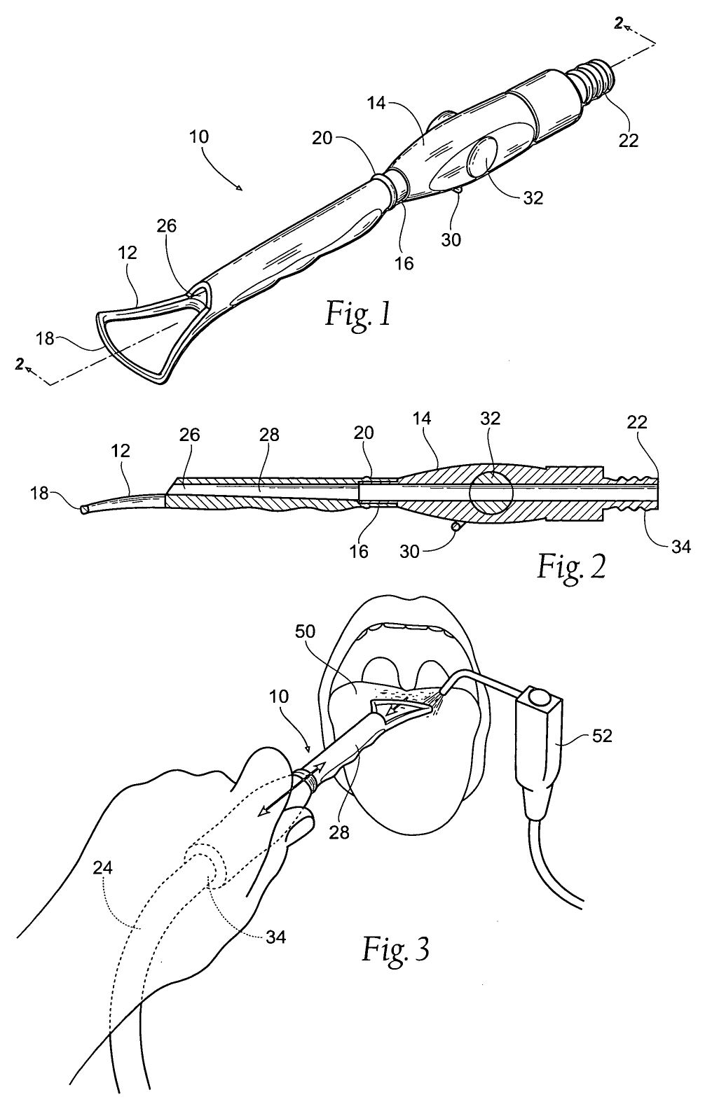

FIG. 1 provides a perspective view of a tongue scraper 10 according to the present invention. The tongue scraper generally comprises a tongue scraping portion 12 and a hand piece 14. The tongue scraping portion 12 has a proximal end 16 and a distal end 18. Generally located between the proximal end 16 and the distal end 18 is an opening 26, which will be discussed with more respect to FIGS. 2 and 3. The hand piece 14 generally comprises a first end 20 and a second end 22. The first end 20 of the hand piece 14 mates with the proximal end 16 of the tongue scraping portion 12. The tongue scraping portion 12 can be designed to be permanently secured to the hand piece 14 or could be removably securable to the hand piece 14, such as by threading or press fitting the tongue scraping portion 12 onto the hand piece 14. The second end 22 is connected to a conduit 24 (see FIG. 3, shown in phantom) that is connected to a vacuum (not shown).

Referring to FIG. 2, a cross-sectional view of the tongue scraper 10 is shown. The hand piece 14 comprises a fluid passageway 28 extending from the second end 22, to the first end 20, and ending at the opening 26. The fluid passageway 28 provides a conduit or pathway that will allow suction to be applied to a tongue when in operation and also allows a pathway for removal of debris from the tongue (see FIG. 3). A lever 30 located on the hand piece 14 is connected to a valve 32 located within the fluid passageway 28 to provide means for controlling fluid flow through the fluid passageway 28. Other devices and configurations could be used as fluid control means for the device. The fluid passageway 28 is in fluid communication with the conduit 24 at the second end 22 of the hand piece 14, with connection means 34 securing the second end 22 with the conduit 24, removably or permanently. The arrangement provides a fluid tight pathway from the vacuum source to the first end 20 of the hand piece 14, which provides the unique combination of a tongue cleaner and aspiration device.

FIG. 3 demonstrates the tongue scraper 10 being used on a person's tongue 50. The tongue scraper 10 is moved back and forth over the tongue 50, thereby scraping debris off of the tongue 50. Though not necessary, a fluid delivery device 52 provides water or another dental solution onto the tongue 50 to assist in the scraping/debriding process. The removed debris and the water will be removed through the passageway 28 and conduit 24 by way of the vacuum source connected to the tongue scraper 10. The scraper 10 allows for an efficient manner of cleaning the tongue. Furthermore, the tongue scraper 10 can be utilized by clinicians, dentists, and dental hygienists at a dentist's office or other similar setting. Previous tongue scrapers did not take advantage of using vacuum devices and the like, as previous scrapers were developed for home use. The present scraper provides a unique tongue scraper that will enhance cleaning processes for the mouth and the tongue for a dentist or dental hygienist.

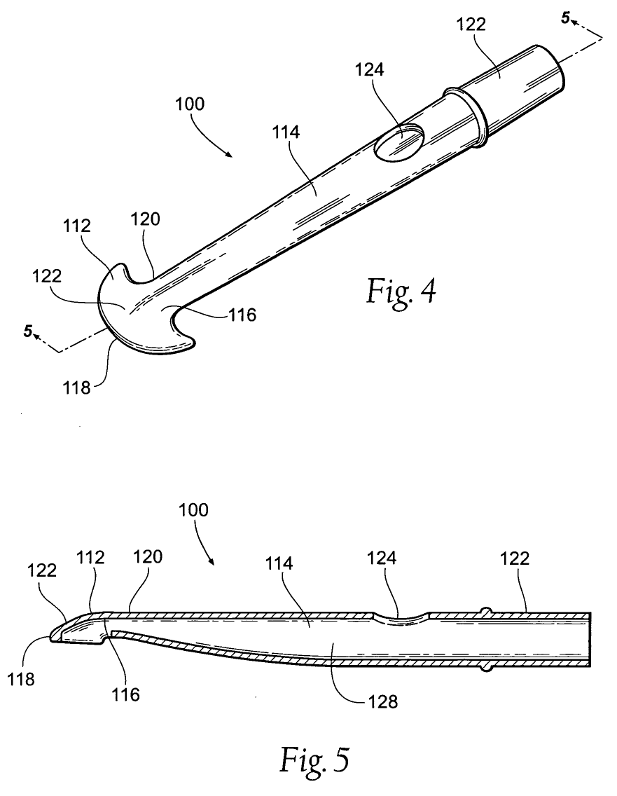

FIGS. 4 and 5 provides an alternate embodiment 100 of the tongue scraper. The tongue scraper 100 generally comprises a tongue scraping portion 112 and a hand piece 114. The tongue scraping portion 112 has a proximal end 116 and a distal end 118. The hand piece 114 generally comprises a first end 120 and a second end 122, with the first end 120 mating with the proximal end 116 of the tongue scraping portion 112. As with the previous embodiment 10, the tongue scraping portion 112 and the hand piece 114 could be designed or molded as a single piece, or could be separate sections that are mated with one another.

Still referring to FIGS. 4 and 5, the hand piece 114 further comprises a fluid pathway 128 that allows debris to be passed from the tongue scraping portion 112. The tongue scraping portion 112 also has an upper surface 122 that will be used to assist in retaining debris before it is passed to and through the hand piece 114. To further regulate the suction being passed through the hand piece, an air hole 124 is located on the hand piece 114. When scraping a person's tongue, the air hole 124 can be left open, to minimize suction on the tongue. When the scraped debris is to be removed, the user can cover the air hole 124, and the fluid pathway 128 will be able to effectively remove the debris. The user can cover the air hole 124 with a thumb while grabbing the device 100, or another piece of material could be used to cover the air hole 124. It is also possible that other means for regulating the vacuum flow could be incorporated into the device 100, such as the valve 32 shown in FIGS. 1 and 2.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I/We claim:

1. A tongue scraper comprising:
a tongue scraping portion;
a hand piece connected to said tongue scraping portion;
a fluid passageway located in said hand piece; and
means for connecting said fluid passageway to a vacuum device.

2. The tongue scraper according to claim 1 further comprising means for controlling fluid flow through said fluid passageway.

3. The tongue scraper according to claim 1 wherein said tongue scraping portion is removable from said hand piece.

4. The tongue scraper according to claim 1 wherein said tongue scraper is autoclavable.

5. A tongue scraper for use with an external vacuum device, said tongue scraper comprising:
a tongue scraper portion;
an elongated hand piece connected having a first end and a second end, said first end connected to said tongue scraper portion;
a fluid passageway located within said hand piece, said fluid passageway extending from said first end to said second end of said hand piece; and
said second end providing means for fluidly securing said fluid passageway to said vacuum device.

6. The tongue scraper according to claim 5 further comprising control means for controlling fluid flow through said fluid passageway.

7. The tongue scraper according to claim 5 wherein said tongue scraper portion is removably securable to said hand piece.

8. A tongue scraper for use with an external vacuum device, said tongue scraper comprising:
a tongue scraper portion having an upper surface;
an elongated hand piece connected having a first end and a second end, said first end connected to said tongue scraper portion;
an opening located in said hand piece;
a fluid passageway located within said hand piece, said fluid passageway extending from said first end to said second end of said hand piece; and
said second end providing means for fluidly securing said fluid passageway to said vacuum device.

9. The tongue scraper according to claim 8, wherein said tongue scraper portion and said hand piece are a unitary piece.
